(19) United States
(12) Patent Application Publication
(10) Pub. No.: US 2011/0040337 A1
(43) Pub. Date: Feb. 17, 2011

(54) SCREW FOR STABILIZING A BONE FRACTURE AND RELATED KIT

(76) Inventor: Piero Budassi, Cremona (IT)

Correspondence Address: HEDMAN & COSTIGAN, P.C., 1230 AVENUE OF THE AMERICAS, 7th floor, NEW YORK, NY, 10020, US

(21) Appl. No.: 12/734,602

(22) PCT Filed: Nov. 10, 2008
(86) PCT No.: PCT/IT2008/000703

§ 371 Date: Oct. 5, 2010

(30) Foreign Application Priority Data
Nov. 13, 2007 (IT) RM2007A000590

(51) Int. Cl. A61B 17/86; A61B 17/58

(52) U.S. Cl. 606/305; 606/93

(57) ABSTRACT

The present invention relates to a screw (1) for stabilization of a bone fracture having a longitudinal body (2), with a lateral surface and a first and a second end (4, 6), said second end being insertable within the bone tissue (10), and a head (5) coupled with said first end (4) of the body (2); said screw being characterized in that it has a main channel (7) passing through said body (2) and said head (5), provided along its longitudinal axis (8); and in that said body (2) has one or more through holes (9) on said lateral surface, communicating with said main channel (7), said main channel (7) and said one or more through holes (9) are suitable to permit passage there through of bone cement and/or biologically active substances that can be introduced through said head (5).

Present invention further relates to a kit for stabilization of fractures.

The present invention relates to a screw for stabilizing a bone fracture and to a related kit.

More specifically, the invention concerns a screw for stabilizing bone fractures after their reduction, permitting employing known stabilization surgical techniques even on osteoporotic bone tissues.

As it is well known, at present the use of screws for stabilization of fractures is widely employed in the traumatology practice.

As it is well known to those skilled in the art, best stabilization effects employing screws depends on health of bone tissue wherein said screws must be implanted. In fact, mechanical resistance of final screw-bone assembly just depends on conditions of the latter determining its containment efficiency.

Therefore, it is well evident that higher is resistance of bone tissue of the subject to be treated, higher is possibility of a good stabilization of fracture and of a total recovery of the limb.

As it is well known, osteoporosis represents a frequent evolution of bone condition during the life of a person, and it is often the lesion most often causing fractures in elderly people and particularly in females.

Therefore, it is particularly difficult applying a synthesis by screws to a patient suffering of the above problem. In fact, large porosity of bone fragments, often reduced to empty shells, does not permit a good strength of screw.

Many different solutions have been suggested until now in order to permit application of screws on a fragile bone tissue. Among these it is possible mentioning the use of angular strength plates or of synthesis means having an intrinsic stability.

Anyway, solution giving the best results is that providing filling the osteoporotic “cavity” with acrylic cement. And then fixing the screw on the same. Mechanical strength of implant is moved from screw-bone interface to the bone cement interface, thus conferring to the obtained assembly a higher resistance.

Technical problem faced of the above solution is due to the fact that often it is difficult implanting one or more screws within cement after having injected bone tissue, jeopardizing the same stabilization of fracture. Moreover, once hardened bone cement, positioning of screws can be difficult and not precise.

Moreover, above technique does not permit further treatments of bone tissue after having positioned screws, for example by biologically active substances, permitting a quicker healing of lesion.

In view of the above, it is object of the present invention that of optimizing distribution of bone cement about synthesis means, thus simplifying surgical technique, suggesting the use of a device or screw that can be implanted providing support to a cementification and/or biologically substances distribution system within its contiguity. In other words, present invention aims providing primary stability of a synthesis implant on a porotic bone and/or promoting diffusion of biologically active substances within the fracture seat after having implanted screws.

It is therefore specific object of the present invention a screw for stabilization of a bone fracture having a longitudinal body, with a lateral surface and a first and a second end, said second end being insertable within the bone tissue, and a head coupled with said first end of the body; said screw being characterized in that it has a main channel passing through said body and said head, provided along its longitudinal axis; and in that said body has one or more through holes on said lateral surface, communicating with said main channel, said main channel and said one or more through holes are suitable to permit passage there through of bone cement and/or biologically active substances that can be introduced through said head.

Always according to the invention, said body can comprise a threading on the lateral surface, and said through holes can be placed among the spire of said threading.

Still according to the invention, said body can be knurled.

Furthermore, according to the invention, said body can have a prismatic shape, particularly a cylindrical shape.

Advantageously, according to the invention, said head can comprise removable couplings with an injection device for injecting said bone cement and/or biologically active substances, such as a syringe.

Preferably, according to the invention, said coupling means comprise a conical body or a screwed coupling.

Always according to the invention, said head can comprise blocking fins.

Still according to the invention, said screw can be slidably coupled with a guide wire by the insertion of the latter within said through channel; said wire guide can be inserted within bone tissue.

Furthermore according to the invention, said screw can comprise a first closure element that can be inserted within said main channel for closing said main channel in correspondence of said second end; and a second closure element that can be coupled with said head.

Advantageously according to the invention, said second end can be flared.

Always according to the invention said screw can comprise an injection cannula that can be removably inserted within said main channel so as said bone cement and/or biologically active substance is introduced from said injection device, so that by moving said injection cannula injection of said bone cement and/or biologically active substance through said holes along said main channel can be adjusted.

It is further object of the present invention a kit of stabilization of fractures comprising one or more screws of the above kind; one or more guide wires that can be introduced within the bone tissue; and a bone cement and/or biologically active substance injection device, that can be mechanically removably coupled with the head of said screw for injection of bone cement and/or biologically active substance and subsequent passage of same through said main channel and said one or more through holes.

Always according to the invention said injection device can be a syringe.

Still according to the invention said kit can comprise an injection cannula that can be removably inserted within said main channel of said screw.

Furthermore, according to the invention, said kit can comprise an introduction tool having a through channel and an ergonomic shape, said tool being coupable with said screw head.

Advantageously, according to the invention, said injection cannula can be inserted within said channel of said introduction tool.

The present invention will be now described, for illustrative and not limitative purposes, according to its preferred embodiments, with particular reference to the figures of the enclosed drawings, wherein:

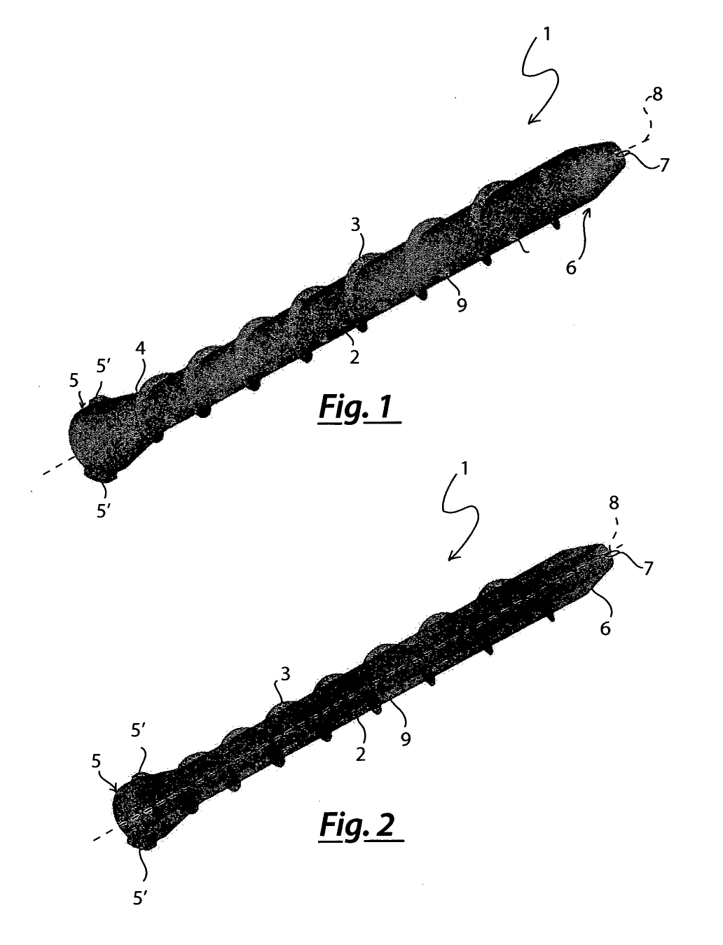

FIG. 1 shows a perspective view for stabilization of a bone fracture according to the present invention;

FIG. 2 shows a transparent perspective view of screw for stabilization of a bone fracture according to FIG. 1;

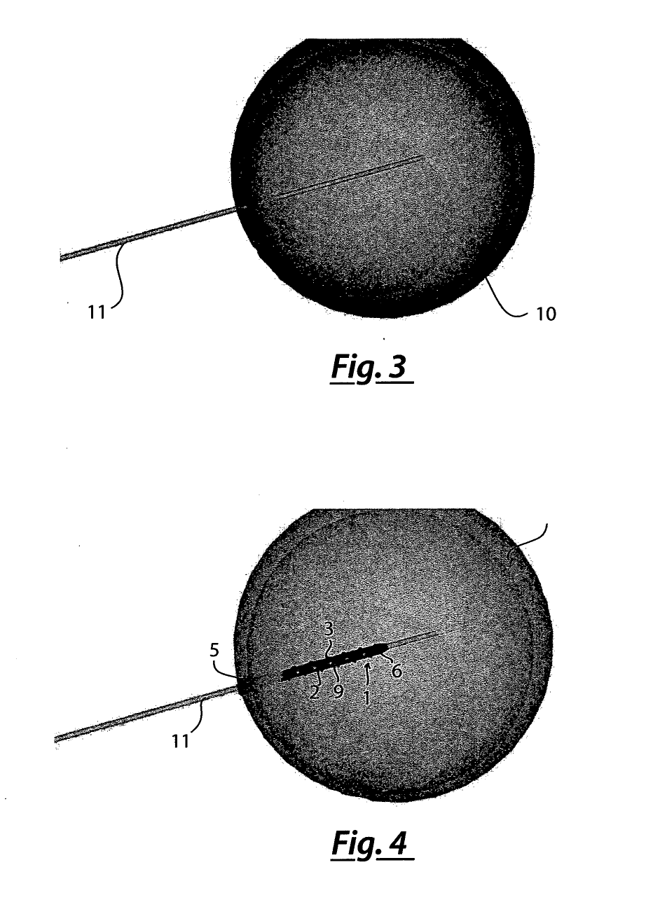

FIG. 3 shows introduction of a guide wire within the bone tissue;

FIG. 4 shows implantation of a screw according to the present invention within bone tissue by the guide wire of FIG. 3;

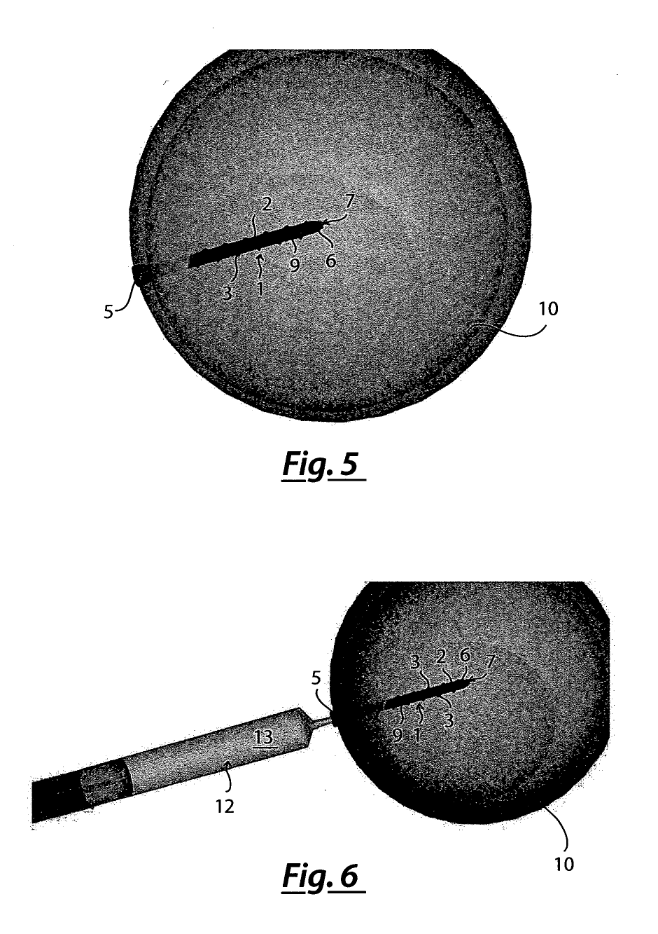

FIG. 5 shows implant of screw according to FIG. 4 from which it is extracted the guide wire into bone tissue;

FIG. 6 shows coupling of a bone cement injection device coupled with the head of the screw according to the present invention;

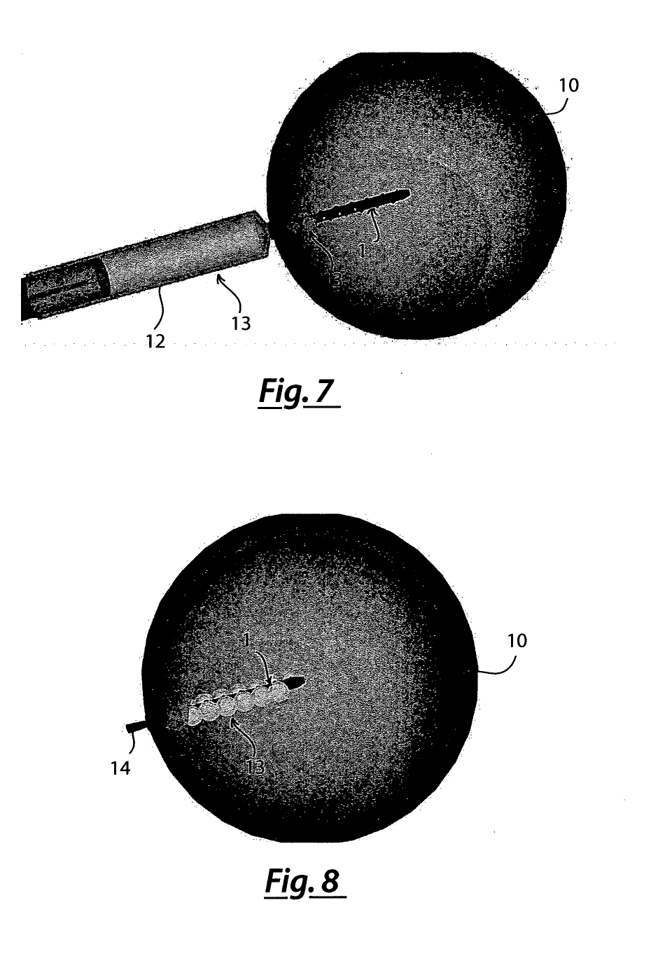

FIG. 7 shows injection of bone cement and screw according to the present invention;

FIG. 8 shows injection of bone cement within bone tissue within surrounding volume of the screw according to the present invention;

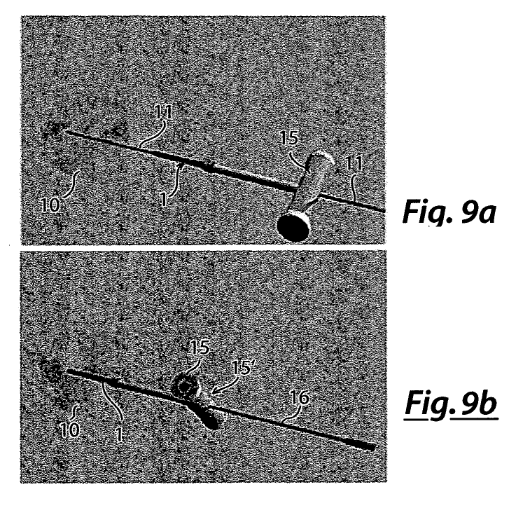

FIGS. 9a and 9b show implantation of the screw according to a further embodiment of the present invention;

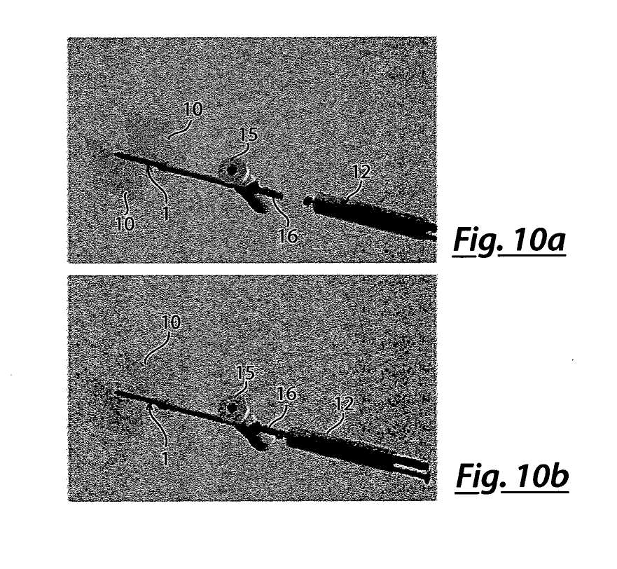

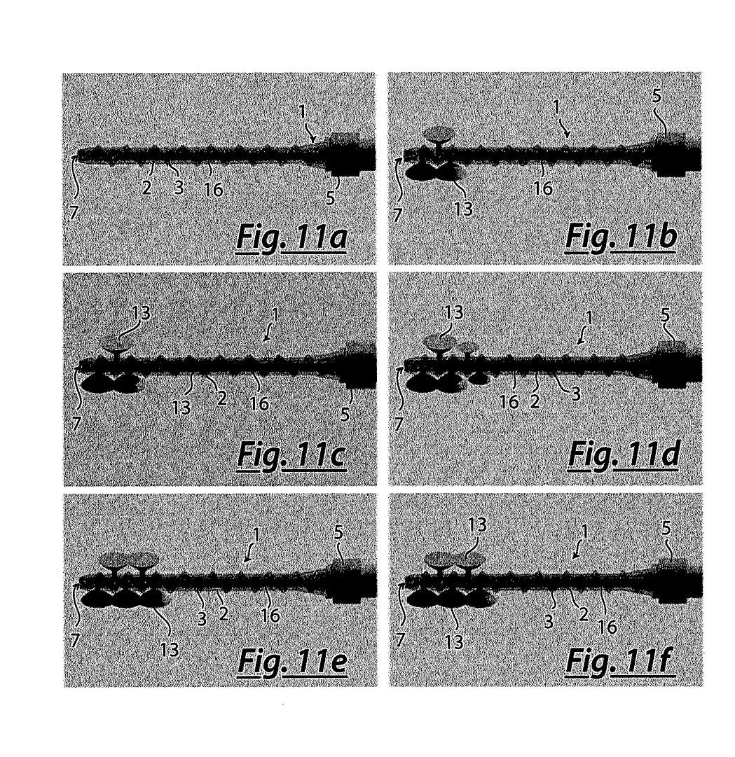

FIGS. 10a and 10b show coupling of an injection device of an introduction tool according to the further embodiment of the present invention; and

FIGS. 11a-11f show a transparent view of the screw according to the further embodiment of the present invention in the different injection steps of bone cement within surrounding volume.

In the different figures similar parts will be indicated by the same reference numbers.

Making reference to FIGS. 1 and 2, it is possible observing a screw 1 for stabilization of a bone fracture according to the present invention, comprised of a bone 2, on the outer surface of which it is provided a threading 3.

Said body 2 has a first end 4, to which a head 5 is coupled, and a second flared end 6.

Said screw 1 also provides a main channel 7. Said main channel 7 passes through said body 2 and through said head 5, along the longitudinal axis 8 of the same screw 1.

Screw 1 according to the invention also has a plurality of through holes 9 communicating with said main channel 7, obtained on the lateral surface of said body 2. Said through holes 9 can be pluri-lateral holes, i.e. uniformly provided all along the outer surface of screw 1, or it can be mono-lateral, i.e. aligned.

Body 2 can have a prismatic shape, and preferably, as shown in the present embodiment, a cylindrical shape permitting, in cooperation with threading 3, a quick introduction within the same bone tissue.

Said head 5 has above a bevel gear pair (not shown in the figures) suitable to permit a mechanical removable coupling for coupling with an injection device, the function of which will be better described in the following. Finally, said head 5 comprises blocking fins 5′.

As it can be observed, said screw 1 is canalized and can be inserted within bone tissue, as it will be better described in the following.

FIGS. 3-8 show the use mode of screw 1 according to the present invention.

One or more guide wires 11 are introduced within fractured and/or osteoporotic bone tissue 10 for stabilization of a bone fracture, to individuate access paths for synthesis means and to realign possible fractured bone stumps (FIG. 3).

Then, said guide wire 11 is inserted within main channel 7 of screw 1. Then, the same screw is inserted within said bone tissue 10. Only the head 5 of screw 1 remains outside the cortical bone wall (the outer wall of the bone), said head being maintained in position by fins 5′ (FIG. 4).

Guide wire 11 is then extracted from said screw 1. Screw 1 is shown in FIG. 5 after having withdrawn guide wire 11.

After extraction of said guide wire 11 from bone tissue 10 within main channel 7, a first closure element (not shown in the figure) is introduced, said closure element sliding will obstruct the opening obtained on second end 6 of said body 2. Then, by conical or threaded coupling, screw 1 is coupled with an injection instrument, such as a syringe, containing different substances, such as bone cement and/or biologically active substances (FIGS. 6).

FIGS. 7 and 8 show that said bone cement and/or biologically active substances 13 is injected within bone tissue 10, filling interspaces caused by fracture or bone porosity.

Once bone cement 13 is hardened, stabilization of bone fracture is obtained.

At the end of the injection, a second closure element 14 is fixed on head.

As example of substances 13 that can be injected by injection device 12 they can be mentioned: bone cement, particularly acrylic cement, hematic extracts and/or growth factors with stamina cells. When said substances start flowing within the screw 1, they exit through holes 9 within bone tissue 10, filling at the same time the interspaces created by fracture or porosity caused by osteoporosis (FIGS. 7 and 8).

In a further embodiment, said body 2 can be knurled instead being threaded.

FIGS. 9a and 9b show a further embodiment of the present invention, wherein it is shown a screw 1, head 5 is coupled with an introduction tool 15, having an ergonomic shape for easily screwing said screw 1 within bone tissue 10, and having a through channel 15′. Screw 1 can be inserted within bone tissue 10 by a guide wire 11, passing through said channel 15 of said introduction tool 15.

Then, once inserted screw 1 within bone tissue 10, guide wire 11 is extracted from screw 1 and insertion tool 15. Then, an injection cannula 16 is inserted, or it is inserted beforehand, both through channel 15′ of introduction tool 15 and screw 1, along its own main channel 7. Said injection cannula 16 is movable along said main channel 7 of the screw 1.

One end of said injection cannula is coupled with the injection device 12 (e.g. syringe) while other end reaches inside of screw 1 along the main channel 7, as it is observed from FIGS. 10a and 10b.

Injection device 12 is in this case provided with a graduate system (reference lines obtained by laser marking) to indicate injection depth (read at the base of introduction tool 15) permitting alignment of an end within injection cannula 16 with a pair of holes 9.

This system permits making a localized injection within interested zones or to obtain a homogeneous distribution of the injected means from holes 9, as it can be observed from FIGS. 11a-11f.

Rearward injection mode has been designed for permitting a homogeneous injection of a substance, such as bone cement and/or biologically active substances. Lacking injection cannula 16, and maintaining uniform hole dimension 9, injected fluid would follow path with the minimum resistance (i.e. outflow would be from the first available holes 9).

With a rearward injection system it is possible aligning the end of the injection cannula 16 with distal holes 9 of screw 1, and the injecting the wished amount, then retracting injection cannula 16 with following reference line and then making a new injection thus in correspondence of the following holes.

For example, making reference to FIGS. 11a-11f, three holes of screw 1 are filled in with cement/osteoinductive substances during the first injection, but they could even be only two holes. Then, retracting injection cannula 16, bone tissue 10 parts can be filled in corresponding to the following holes 10.

An advantage of the present invention is that introduction of the above substances permits on one side consolidating fractured tissues about a bearing element, i.e. screw 1, and on the other hand the same substances permit stimulating growth of tissue, improving its performances.

Present invention has been described for illustrative and not limitative purposes with reference to preferred embodiments, but it is understood that variations and/or modifications can be introduced without departing from the relevant scope defined in the enclosed claims.

1. Screw (1) for stabilization of a bone fracture having a longitudinal body (2), with a lateral surface and a first and a second end (4, 6), said second end being insertable within the bone tissue (10), and a head (5) coupled with said first end (4) of the body (2); said screw has a main channel (7) passing through said body (2) and said head (5), provided along its longitudinal axis (8); said body (2) has one or more through holes (9) on said lateral surface, communicating with said main channel (7), said main channel (7) and said one or more through holes (9) being suitable to permit passage there through of bone cement and/or biologically active substances that can be introduced through said head (5).

2. Screw (1) according to claim 1, characterized in that said body (2) comprises a threading (3) on the lateral surface and said through holes (0) are placed on the spire of the threading.

3. (canceled)

4. Screw (1) according to claim 1, characterized in that said body is knurled.

5. Screw (1) according to claim 1, characterized in that said body (2) has a prismatic shape, particularly a cylindrical shape.

6. Screw (1) according to claim 1, characterized in that said head (5) comprises removable couplings with an injection device (12) for injecting said bone cement and/or biologically active substances, such as a syringe.

7. Screw (1) according to claim 6, characterized in that said coupling means comprise a conical body.

8. Screw (1) according to claim 6, characterized in that said coupling means comprise a screwed coupling.

9. Screw (1) according to claim 1, characterized in that said head (5) comprises blocking fins (5′).

10. Screw (1) according to claim 1, characterized in that it is slidably coupled with a guide wire (11) by the insertion of the latter within said through channel (7); said wire guide (11) can be inserted within bone tissue (10).

11. Screw (1) according to claim 1, characterized in that it comprises a first closure element, that can be inserted within said main channel (7) for closing said main channel (7) in correspondence of said second end (6).

12. Screw (1) according to claim 1, characterized in that said second end (6) is flared.

13. Screw system characterized in that it comprises a screw as defined in claim 1 an injection device (12), for injecting said bone cement and/or biologically active substances, and
an injection cannula (16) that can be removably inserted within said main channel (7)of said screw, said bone cement and/or biologically active substance introduced by injection device (12) through said cannula (16), so that by moving said injection cannula (16), the injection of said bone cement and/or biologically active substance through said holes (9) can be adjusted along said main channel (7).

14. Kit for stabilization of fractures comprising one or more screws (1) as defined in claim 1; one or more guide wires (11) that can be introduced within the bone tissue; and a bone cement and/or biologically active substance (13) injection device (12), that can be mechanically removably coupled with the head (5) of said screw (1) for injection of bone cement and/or biologically active substance (13) and subsequent passage of same through said main channel (7) and said one or more through holes (9).

15. Kit according to claim 14, characterized in that said injection device is a syringe (12).

16. Kit according to claim 14, characterized in that it comprises an injection cannula (16) that can be removably inserted within said main channel (7) of said screw (1).

17. Kit according to claim 14, characterized in that it comprises an introduction tool (15) having a through channel (15′) and an ergonomic shape, said tool being coupable with said screw (1) head (5).

18. Kit according to claim 17, characterized in that said injection device is a syringe (12) and injection cannula (16) can be inserted within said channel (15′) of said introduction tool (15).
